(19) United States
(12) Patent Application Publication
(10) Pub. No.: US 2015/0184215 A1
(43) Pub. Date: Jul. 2, 2015

(54) DEVELOPMENT OF THE SOLUBLE RECOMBINANT CRM197 PRODUCTION BY E. COLI

(71) Applicant: Development Center for Biotechnology, New Taipei City (TW)

(72) Inventors: Yu-Shen Hsu, New Taipei City (TW); Show-Shan Sheu, New Taipei City (TW); Bo-Chin Lei, New Taipei City (TW); Tsan-Hui Wu, New Taipei City (TW)

(21) Appl. No.: 14/584,836

(22) Filed: Dec. 29, 2014

Related U.S. Application Data
(60) Provisional application No. 61/921,293, filed on Dec. 27, 2013.

(51) Int. Cl. C12P 21/00; C07K 1/20; C07K 14/34; C07K 1/18; C07K 1/22

(52) U.S. Cl. CPC C12P 21/00; C07K 1/18; C07K 1/22; C07K 14/34; C07K 1/20

(57) ABSTRACT

A method for recombinant production of a CRM197 protein includes culturing a recombinant Escherichia coli cell to produce said CRM197 protein, and isolating said CRM197 protein. The recombinant Escherichia coli cell includes an expression vector, which contains a nucleic acid molecule that encodes a fusion protein that includes an E. coli periplasmic signal peptide at N terminal and the CRM197 at C terminal. The CRM197 is encoded by a polynucleotide having the sequence of SEQ ID NO: 1. The E. coli periplasmic signal peptide comprise a pelB leader sequence. The nucleic acid molecule comprises the sequence of SEQ ID NO:2. The Escherichia coli cell is BL21(DE3)pLysS.

### FIELD OF THE INVENTION

The present invention relates to the field of the production of proteins of pharmacological interest, particular, it relates to the production of CRM197 in Escherichia coli, and to a method for the isolation and purification of the protein CRM197.

### BACKGROUND OF INVENTION

CRM197 (cross-reacting material 197; 58 kDa) is a variant of diphtheria toxin (DTx). CRM197 contains a single amino-acid mutation (G52E; a glycine-glutamic acid substitution at position 52) in DTx. This mutation reduces its toxicity, but retains the inflammatory and immunostimulant properties of DTx (Uchida T. et al, 1973; Giannini G. et al, 1984). Therefore, CRM197 may be used in the preparation of conjugated vaccines.

Similar to DTx, CR_M197 comprises two domains, A and B, joined by a disulfide bond. The A domain (21 kDa) contains a catalytic domain, while the B domain (37 kDa) is composed of two subdomains: one for binding to the cell receptor and the other for translocation (Gill D. M. et al, 1971; Uchida T. et al, 1973), The single amino-acid mutation does not alter the structure and function of the B domain. Therefore, CRM197 retains the ability to bind to cell surface receptor, HB-EGF (heparin binding epidermal growth factor). The binding allows CRM197 to translocate to the inside of a cell by endocytosis.

In the cells, the low pH in the endosomes induces a conformational change, resulting in the insertion of the B domain into the membrane, which is followed by translocation of the A domain into the cytosol (Papini E. et al, 1993; Cabiaux V. et al, 1997). Before the A domain can be released into the cytosol, a peptide bond between the two domains A and B is by a protease and disulfide bridge is reduced an broken. The released A domain becomes catalytically active, while the whole protein is inactive (Gill D. M. et al, 1971).

The released A domain of DTx is an ADP-ribosyl transferase and can catalyze the transfer of an ADP-ribose group from NAD to the elongation factor 2 (EF-2). The ADP-robosylated EF-2 is inactive, resulting in the disruption of protein synthesis (Honjio T. et al, 1971). In addition, the active A domain also has a non-specifically endonuclease activity, which can degrade DNA (Giannini G. et al, 1984).

CRM197 and other non-toxic variants are typically produced using lysogenic cultures of Corynebacterium diphtheriae infected with phages that contain in their genomes a mutant tox gene that encodes the diphtheria toxin (DTx). The diphtheria toxin variants may be induced to secrete into the culture medium and purified (Cox J., 1975). However, production of CRM197 from single lysogenic strains of Corynebacterium was not economical. To increase the production of CRM197, double and triple lysogenic mutants that contain two or three tox genes integrated in the chromosome have been used (Rappuoli R. et al, 1983; Rappuoli R., 1983).

For example, U.S. Pat. No. 4,925,792, issued to Rappuoli, describes a process for the production of DTx variants using a strain of Corynebacterium with two copies of the mutated tox gene integrated in the chromosome. Production of CRM197 using this system, however, is not very convenient. CRM197 accumulates in the culture medium throughout the logarithmic growth phase. However, there is a considerable decline in the yield in the later phase due probably to proteolysis (U.S. Pat. No. 4,925,792).

Stefan first reported the expression of CRM197 using E. coli (Journal of Biotechnology 2011, 156:245-252 or US2012/0128727A1). The expression of the whole CRM197 protein in E. coli has not been reported since Stefan's report. In Stefan's report provided a method for the production and purification of full-length rCRM197 in E. coli. However, In Stefan's method, the recombinant CRM197 was produced as a fusion protein associated with a short tag, and this produced rCRM197his in E. coli was presented in the insoluble protein fraction. Several purification steps and an additional cleavage of the tag were necessary to obtain the recombinant CRM197.

### SUMMARY OF INVENTION

Embodiments of this invention relate to methods for producing the soluble, full-length recombinant CRM197 in E. coli with high yields. The expressed recombinant CRM197 has been checked with anti-CRM197 antibody in Western blot analysis and the amino acid sequence of recombinant CRM197 was confirmed with peptide mapping analysis.

In one aspect, embodiments of the invention relate to expression vectors. An expression vector in accordance with one embodiment of the invention comprises a nucleic acid molecule that encodes a fusion protein that includes an E. coli periplasmic signal peptide at N terminal and the CRM197 at C terminal.

In one aspect, embodiments of the invention relate to recombinant Escherichia coli cell includes an expression vector, which contains a nucleic acid molecule that encodes a fusion protein that includes an E. coli periplasmic signal peptide at N terminal and the CRM197 at C terminal.

In one aspect, embodiments of the invention relate to methods for recombinant production of a CRM197 protein. A method in accordance with one embodiment of the invention includes culturing a recombinant Escherichia coli cell to produce said CRM197 protein, and isolating said CRM197 protein. The recombinant Escherichia coli cell includes an expression vector, which contains a nucleic acid molecule that encodes a fusion protein that includes an E. coli periplasmic signal peptide at N terminal and the CRM197 at C terminal.

In accordance with any one of the above embodiments of the invention, the CRM197 may be encoded by a polynucleotide having the sequence of SEQ ID NO: 1. The E. coli periplasmic signal peptide may comprise a pelB leader sequence. The nucleic acid molecule may comprise the sequence of SEQ ID NO:2. The Escherichia coli cell may be BL21(DE3)pLysS.

Other aspects of the invention will be apparent in view of the attached drawings and the following description.

### BRIEF DESCRIPTION OF THE DRAWINGS

FIG. 1 shows an optimized sequence encoding CRM 197 optimized for expression in E. coli (SEQ ID NO: 1) in accordance with one embodiment of the invention.

FIG. 2 shows an artificial sequence encoding CRM197 inserted into the pET27b(+) in accordance with one embodiment of the invention. At the 5′ end of the CRM197 coding sequence, there is an oligonucleotide sequence encoding the pelB leader (an N-terminal pelB signal sequence for potential periplasmic localization) (SEQ ID NO: 2). The signal sequence corresponding to the pelB leader is underlined.

FIG. 3 shows SDS-PAGE of protein extracts obtained from BL21DE3pLysS transformed with pET27b-CRM197. Lanes 1: insoluble fraction obtained from the induced cell protein extracts after the removal of soluble fraction. Lane 2: the soluble fraction of total cell protein extracts. The arrow shows the band corresponding to the recombinant CRM197.

FIG. 4 shows Western blot analysis of the soluble fraction of total protein extracts. Lane 1: 1 μg of the commercial product of CRM197 purchased from Sigma. Lane 2: 2 μl of the soluble fraction of total protein extracts from supernatant (total supernatant was 32 ml from 500 ml cultures). To identify the recombinant CRM197 protein, a polyclonal anti-Diphtheria Toxin antibody conjugated with HRP was used with 200-folds dilution (Abcam).

FIG. 5 shows a total lysate of E. coli cells expressed rCRM197 separated on 4-12% Nu page Bis-Tris Gels (Invitrogen, NP0321BOX). Lane 1 is a commercial CRM197 sample purchased from Sigma and lane 2 is the sample from the lysate with rCRM197 expression. The band of rCRM197 about 58 kDa on lane 2 was used to analyze protein sequence by peptide mapping with LC/MS/MS.

FIG. 6 shows protein sequence (SEQ ID NO: 3) of recombinant CRM197 expressed by E. coli was confirmed with peptide mapping analysis.

### DETAILED DESCRIPTION OF THE INVENTION

Embodiments of the invention relate to methods for the production of full-length recombinant CRM197. This protein is widely used as a carrier for polysaccharide conjugated vaccines, because no formaldehyde detoxification is required and its immunogenicity is extremely high.

A method in accordance with embodiments of the invention involves cloning a codon optimized clone into an expression vector for transformation of E. coli. After culture and induction of E. coli as bacterial host under optimal conditions, over-expression of the recombinant CRM197 protein in soluble form was successfully achieved. This method will be helpful for the production and purification of the recombinant CRM197 at the native conformation with cost-effective yields, reduced time, and simple culture medium.

Embodies of the invention will be further illustrated with specific examples set forth below. One skilled in the art would appreciate that these examples are for illustration only an dare not intended to be limiting because variations and modifications are possible without departing from the scope of the invention.

### MATERIALS AND METHODS

### Bacterial Strains and Growth Conditions

E. coli BL21DE3pLysS cells (E. coli B F-dcm ompT hsdS(rB−mB−) gal λ (DE3)[pLysS Camr] (Novagen, Inc.) were used as host for the expression of recombinant CRM197. Cultures were grown in LB standard medium supplemented with kanamycin (30 μg/mL). IPTG (0.5 mM) was added to LB as inducer.

### CRM197 Gene Design

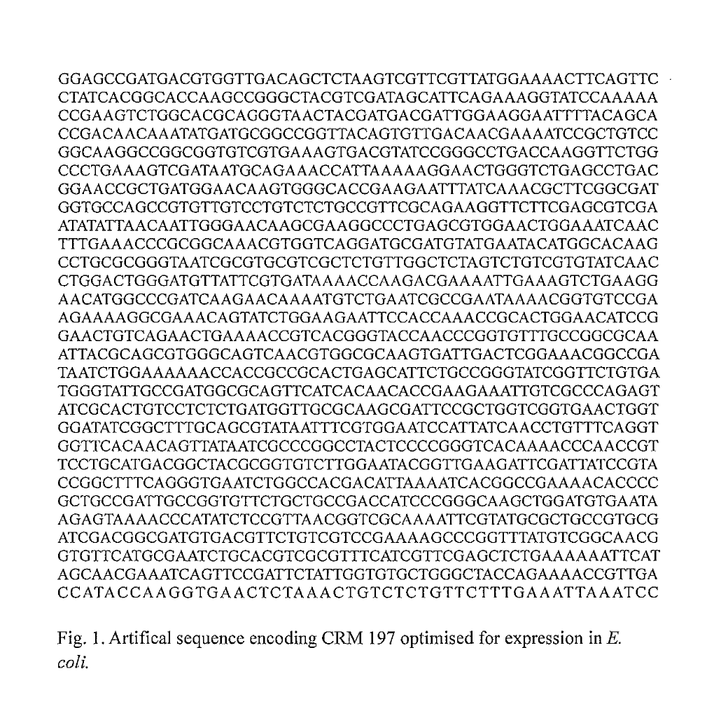

To improve the production of rCRM197, the codons for this protein are optimized. The optimized sequence encoding the rCRM197 is shown in FIG. 1. (SEQ ID NO:1). The nucleotide sequence was optimized for E. coli codon usage by using the GenScript analysis software.

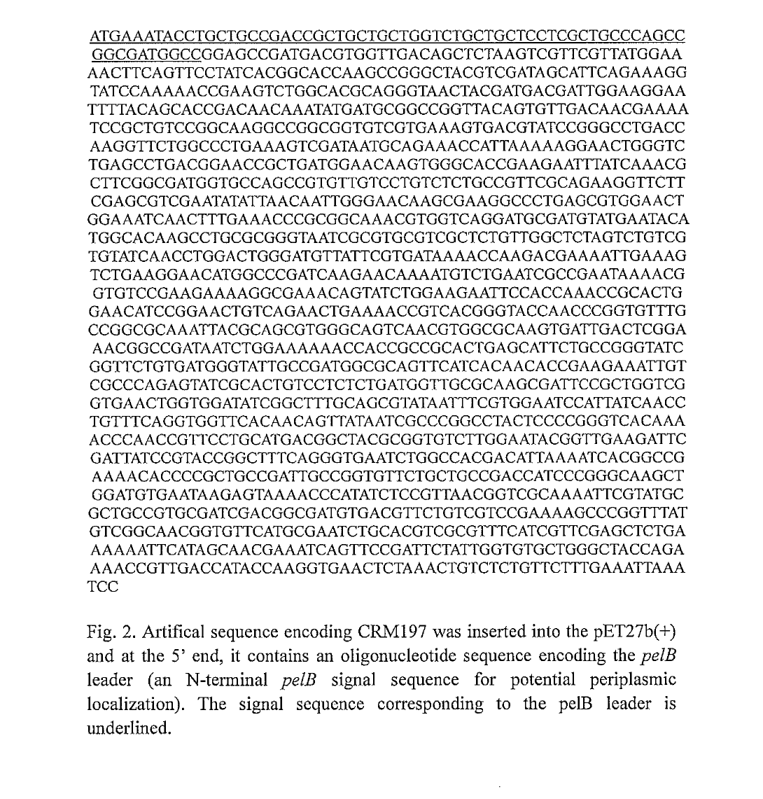

The codon optimized synthetic gene corresponding to CRM197 was then cloned into pET27b(+) vector (Novagen, Inc.) (FIG. 2) at the NdeI and XhoI restriction sites. FIG. 2 shows an artificial sequence encoding CRM197 inserted into the pET27b(+). At the 5′ end, this sequence contains an oligonucleotide sequence encoding the pelB leader (an N-terminal pelB signal sequence for potential periplasmic localization) (SEQ ID NO:2). The signal sequence corresponding to the pelB leader is underlined in FIG. 2.

Using the pelB leader sequence to direct the expressed rCRM197 into the periplasmic space has the advantage that the expressed protein would not be in the reductive environment of cytosol. Instead, the oxidative environment in the periplasmic space would facilitate the disulfide bond formation. This may in turn facilitate the proper folding of the expressed protein, which would remain as soluble forms, not aggregates. Soluble form of rCRM197 would be easier to purify.

Cloning procedures were performed following the standard procedures. About 50 ng of recombinant construct of pET27b(+)-CRM197 was transformed into E. coli cells DI-15, and transformants were selected on LB agar plate supplemented with 30 μg/ml kanamycin. The confirmed plasmid was then transformed into expression host strain BL21DE3pLysS.

### Protein Expression

Single colonies of BL21DE3pLysS containing the recombinant constructs pET27b-CRM197 were grown overnight at 37° C. in 2 mL of LB supplemented with 30 μg/ml kanamycin. Cultures were diluted (1:500) in fresh medium and incubated at 37 ° C. under shaking conditions. The inducer (IPTG) was then added to 0.1-1.0 mM (e.g., 0.5 mM) when cell populations reached an OD600 of 0.5 or higher, such as OD600 of 1.0 or higher, 10 or higher, 30 or higher, or 60 or higher. The induction may be performed for any suitable time, such as 10 hours or more.

Aliquots were harvested by centrifugation (4000×g for 15 min) at different time intervals after induction (at 0 h and overnight). To verify the expression of rCRM197, total protein extracts were analyzed on 4-12% Nu page Bis-Tris Gels (Invitrogen, NP0321BOX). Samples were prepared by adding sample buffer 5×(10% SDS, 10 mM 2-mercaptoethanol, 30% glycerol, 0.2 M Tris-HCl pH 6.8, 0.05% bromophenol blue) and boiled for 5 min. After separation, gels were stained with 0.1% Coomassie brilliant blue R-250.

In accordance with some embodiments of the invention, a method of the invention may, for example, comprises: (a) Growing E. coli that harbors a plasmid or an expression vector carrying a gene of CRM197, such as the one described above, in a pre-culture and subsequently fermenting in a main culture; (b) Producing the CRM197 protein from said main culture; wherein said main culture is a fed-batch fermentation comprising a batch phase and a fed-batch phase; (c) culturing the E. coli cells in batch phase with batch media until the cell density higher than the OD600 nm value of 10 or more; (d) culturing the E. coli cells in fed-batch phase with addition of the first feed medium by stepwise feeding rate; and (e) culturing the E. coli cells in fed-batch phase with addition of the second feed medium at a constant feeding rate.

In accordance with any of the above embodiments, the first feed medium may be added at a suitable flow rate with stirring, for example from 100 to 400 ml/hr, with stirring at a speed from 300 to 800 rpm. In accordance with these embodiments, the feeding rate and the stirring speed may be increased stepwise.

In accordance with any of the above embodiments, the second feed medium may be added at a suitable flow rate, for example, from 50 to 300ml/hr, with stirring at a speed from 300 to 800 rpm. In accordance with these embodiments, the feeding rate is constant added and the stirring speed is decreased stepwise.

In accordance with any of the above embodiments, the batch medium may comprise: (a) an organic carbon source, which may be a sugar, such as one selected from glucose, glycerol, fructose, lactose, sucrose, arabinose; galactose, and mannose; (b) an organic nitrogen source, such as one selected from yeast extract and phytone peptone, which is animal source free and present as a component of the media or added to the media during fermentation; and (c) one or more inorganic salts, which may be any suitable inorganic salts known in the art, such as chloride salts, sulfate salts, and phosphate salts.

In accordance with any of the above embodiments, the first feed medium may comprise glucose at a concentration from 150 to 500 g/L and yeast extract at a concentration from 100 to 500 g/L.

In accordance with any of the above embodiments, the second medium comprising glucose at aconcentration from 50 to 400 g/L and yeast extract at a concentration from 50 to 400 g/L.

In accordance with any of the above embodiments, the temperature during induction phase may be shifted down from 37 degree Celsius to a temperature between 32-16 degree Celsius.

### Production

For the production of recombinant CRM197, 500 ml of cultures were grown under the same conditions described above. However, the inducer (IPTG) was added to a concentration of 0.5 mM when the cell populations reached an OD600 of 0.5. After induction, the cultures were incubated at 20° C. under shaking conditions overnight.

As noted above, the optimized expression sequence contains a pelB leader sequence, which will direct the expressed protein into the periplasmic space. This would facilitate the isolation of the desired protein. For example, one can first collect the cells to separate the desired protein from various components in the media. Then, the cells can be lysed to obtain the soluble fraction containing the desired protein.

Accordingly, cells were harvested from the above culture by centrifugation at 4000×g for 20 min, and pellets were suspended in 1/10 of volume of lysis buffer (50 mM Tris-HCl pH 8, 500 mM NaCl, Triton X-100 1%, phenyl methyl sulfonyl fluoride 1 mM). Then, the suspension was microfluidized with a Microfluidizer® by at least 5 cycle passages of the whole volume or fluid. Microfluidizer® is a high shear fluid processor, which is used for cell disruption.

After microfluidization, the supernatant was obtained by centrifugation at 4000×g for 20 min from cellular lysis. Supernatants containing the soluble fraction were collected and may be stored at 4° C. until analysis.

### Purification

In accordance with any of the above embodiments, CRM197 recombinant protein from fermentation using the E. coli cell may be purified using chromatography. In some embodiments, the purification may involve three steps of chromatography purification, which may be performed sequentially. In accordance with any of these embodiments, the first step may involve capture purification, which may be performed by anionic exchange chromatography. The anion exchanger may be diethylaminopropyl (ANX), diethyl-aminoethyl (DEAE), Quaternary aminoethyl (QAE), Quaternary ammonium (Q), positive charge function groups immobilized to the base matrix, or a mixed mode resin coupled with N-benzyln-methyl ethanolamine functional group.

In accordance with embodiments of the invention, second intermediated purification may be performed by hydrophobic interaction chromatography (HIC), which may include butyl or octyl functional groups, wherein the aromatic or aliphatic ligands may be immobilized to the base matrix.

In accordance with embodiments of the invention, third purification may be performed by affinity chromatography. The resins for affinity chromatography may include different functional affinity groups connected to the base matrix. The functional affinity groups may be specified repeating dimer of hexuronic acid and D-glucosamine, or other ligands such as Cibacron Blue 3G, Lysine, metal ion (Zn²⁺, Cu²⁺, Ni²⁺, Co²⁺ ions), lectins, calcium phosphate repeating stretch.

### Characterization of Recombinant CRM197 Protein

To identify the expression of the recombinant CRM197 protein in soluble fraction from cell lysate of the cells transformed with pET27b-CRM197, a Western immunoblotting with anti-Diphtheria Toxin antibody-HRP (Abeam) was performed using 4-12% Nu page Bis-Tris Gels (Invitrogen, NP0321BOX). The amino acid sequence of the expressed recombinant CRM197 was further confirmed by peptide mapping analysis.

### Expression and Analysis of Recombinant CRM197

The codon optimized synthetic gene corresponding to CRM197 (FIG. 1) cloned into pET27b(+) vector was used to obtain an optimized sequence (FIG. 2) for the expression. The expressed recombinant CRM197 was localized in the periplasmic space in E. coli. Considering that CRM197 might be toxic for E. coli, the pET27b(+)-CRM197 vectors were transformed into E. coli BL21DE3pLysS for rCRM197 expression.

After the addition of IPTG to induce BL21DE3pLysS cultures to express rCRM197, cells were harvested by centrifugation. To verify the rCRM197 expressed in the soluble fraction in the periplasmic space, cell pellets were resuspended in 1/10 of volume of lysis buffer (50 mM Tris-HCl pH 8, 500 mM NaCl, Triton X-100 1%, phenyl methyl sulfonyl fluoride 1 mM). Then, the suspension was microfluidized with a microfluidizer by at least 5 cycle passages of the whole volume of fluid. The supernatant was obtained by centrifugation at 4000×g for 20 min from cellular lysis. Supernatants containing the soluble fraction and cellular lysis were collected and analyzed on 4-12% Nu page Bis-Tris Gels (Invitrogen, NP0321BOX).

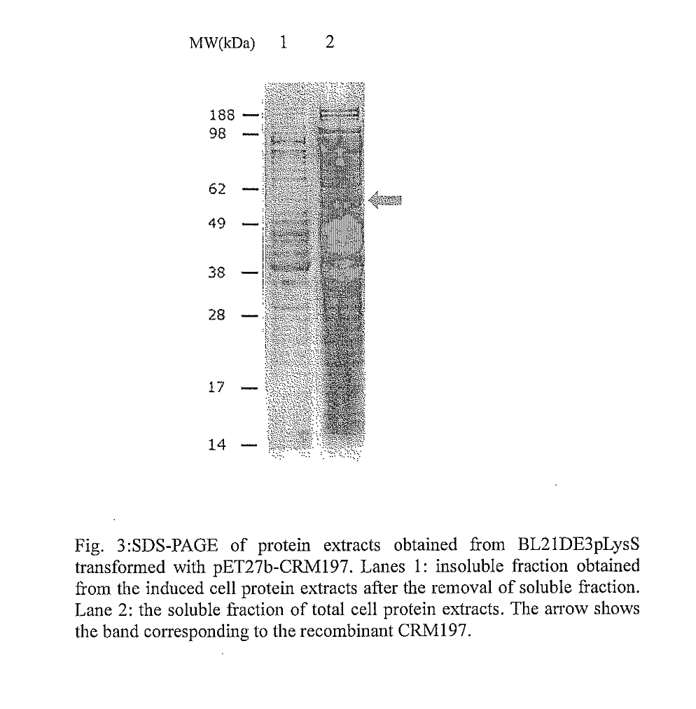

As shown in FIG. 3, lane 1 shows the sample from insoluble cellular lysis, and lane 2 is the sample from supernatant fraction. The arrow shows a band of about 58 kDa corresponding to rCRM197 presents in lane 2. This observation indicates that the rCRM197 is expressed as a soluble protein. The produced rCRM197 was further confirmed by Western blot analysis, by probing of the band with anti-diphtheria toxin antibody-HRP.

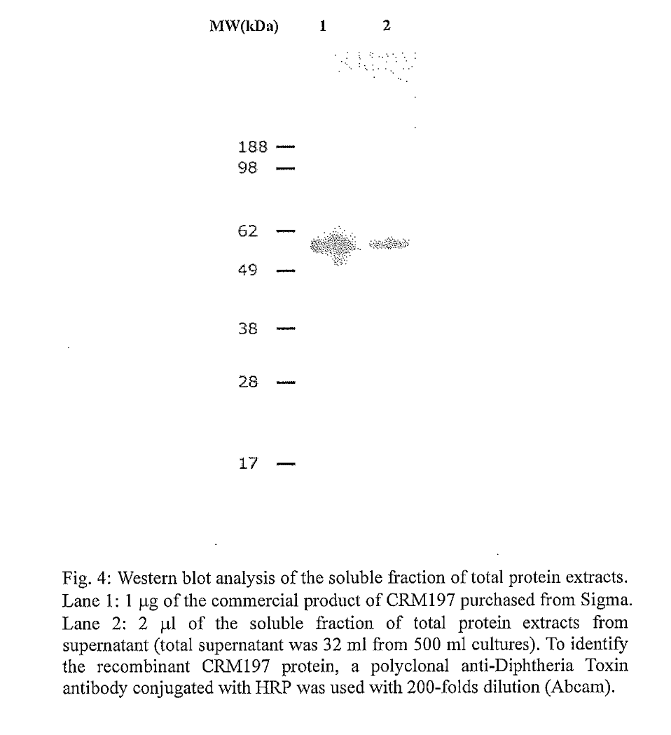

As shown in FIG. 4, lane 1 is a commercial CRM197 sample, which is used as a positive control, and lane 2 is the sample from supernatant fraction from the bacterial expression. In FIG. 4, a band of about 58 kDa observed in lane 2 of FIG. 3 was detected with anti-diphtheria toxin antibody and showed the same molecular weight as commercial CRM197. This result indicates that the expressed protein is the full-length rCRM197.

In FIG. 4, Lane 1 contains 1 μg of the commercial product of CRM197 purchased from Sigma, Lane 2 contains 2 μl of the soluble fraction of total protein extracts from supernatant. The intensities of the bands in Lane 1 and Lane 2 are not that much different. Let's assume a very conservative estimate that the intensity in Lane 2 is about 1/10 of that in Lane 1, then Lane 2 would contain 0.1 μg of rCRM197. The sample in Lane 2 is 2 μl out of a total supernatant of 32 ml obtained from 500 ml cultures. This would give about 1.6 mg of rCRM197 in 500 ml culture, which is quite impressive considering this probably severely underestimates the actual quantity (the band in Lane 2 is actually much more than 1/10 of Lane 1) and that this is expressed in the periplasmic space.

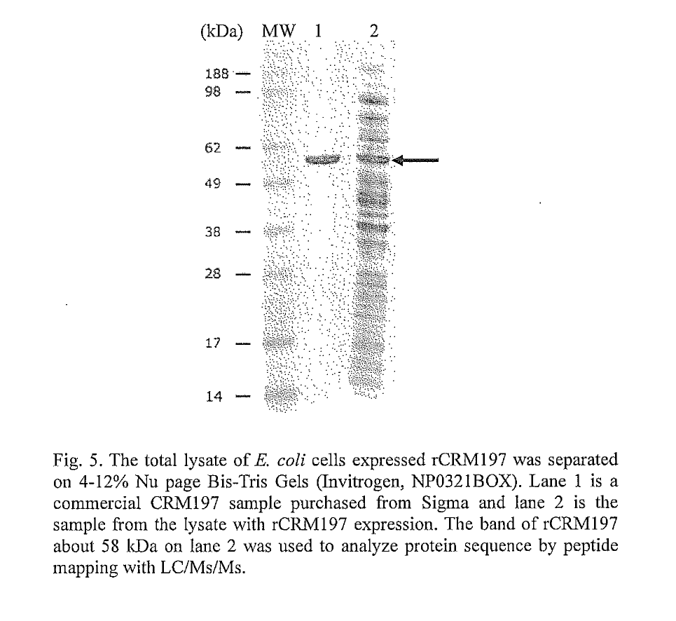

For further identify the rCRM197, the total lysate of E. coli cells expressing rCRM197 was separated on 4-12% Nu page Bis-Tris Gels (Invitrogen, NP0321BOX). As shown in FIG. 5, lane 1 is a commercial CRM197 sample and lane 2 is the sample from the lysate with rCRM197 expression. The expressed rCRM197 in lane 2 is indicated with an arrow. Results shown in FIG. 5 suggest that the expressed rCRM197 is identical to the commercial sample.

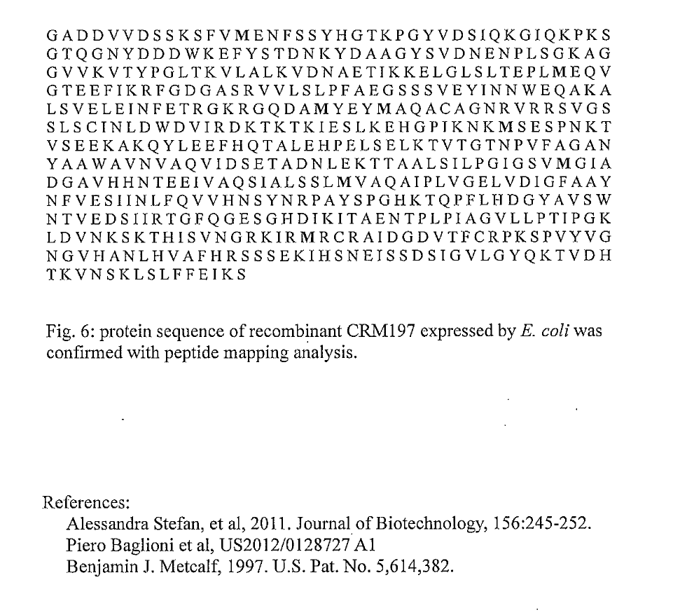

The band of rCRM197 with an apparent molecular weight of about 58 kDa in lane 2 was further analyzed by peptide mapping using LC/MS/MS to identify the protein sequence (performed by Core facility for Protein Structural Analysis, Institute of Biological Chemistry, Academia Sinica, Taiwan). The identified protein sequence is identical to the commercial product of CRM197 purchased from Sigma. The peptide mapping results also show that the pelB signal peptide was removed by E. coli host and didn't show up in the final rCRM197 product. The CRM197 protein sequence is shown in FIG. 6.

The above results clearly indicate that rCRN197 can be produced as soluble proteins using a method of the invention. Furthermore, the rCRM197 protein is expressed in periplasmic space, which facilitates the purification and also facilitates the formation of soluble form of the protein, probably with the correct disulfide bond. In addition to the ease of expression and ease of purification, the yield is also very good. Thus, with methods of the invention, one can easily obtain sufficient amount of relatively pure drCRM197 for various applications.

Advantages of the invention may include one or more of the followings. A method of the invention can produce CRM197 with high yield, much higher than those in the prior art. Embodiments of the invention are easy to implement. The proteins are expressed as soluble form. Purification of the expressed proteins is relatively simple and easy.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### REFERENCES

Alessandra Stefan, et al, 2011. Journal of Biotechnology, 156:245-252.

Piero Baglioni et al, US2012/0128727 A1

Benjamin J. Metcalf, 1997. U.S. Pat. No. 5,614,382.

What is claimed is:

1. An expression vector, comprising a nucleic acid molecule which encodes a fusion protein comprising an E. coli periplasmic signal peptide at N terminal and CRM197 at C terminal, wherein the CRM197 is encoded by a polynucleotide having the sequence of SEQ ID NO: 1.

2. The expression vector of claim 1, wherein the E. coli periplasmic signal peptide comprise a pelB leader sequence.

3. The expression vector of claim 1, wherein the nucleic acid molecule comprises the sequence of SEQ ID NO:2.

4. A recombinant Escherichia coli cell comprising the expression vector of claim 1.

5. The recombinant Escherichia coli cell of claim 4, wherein the Escherichia coli cell is BL21(DE3)pLysS.

6. A method for recombinant production of a CRM197 protein, comprising:
culturing the recombinant Escherichia coli cell to produce said CRM197 protein, and
isolating said CRM197 protein,
wherein the CRM197 protein has the amino acid sequence of SEQ 1D NO: 3.

7. The method of claim 6, wherein the isolating comprises obtaining the CRM197 protein from periplasmic space.

8. The method of claim 6, wherein said method for producing CRM197 in recombinant E. coli cells, comprising:
a. Growing E. coli that harbors a plasmid carrying a gene of CRM197 in a pre-culture and subsequently fermenting in a main culture;
b. Producing the CRM197 protein from said main culture; wherein said main culture is a fed-batch fermentation comprising a batch phase and a fed-batch phase;
c. culturing the E. coli cells in batch phase with batch media until the cell density higher than the OD600 nm value of 10;
d. culturing the E. coli cells in fed-batch phase with addition of the first feed medium by stepwise feeding rate; and
e. culturing the E. coli cells in fed-batch phase with addition of the second feed medium by a constant feeding rate;

9. The method of claim 8, wherein the expression of CRM197 protein is induced by addition of isopropyl β-D-1-thiogalactopyranoside (IPTG).

10. The method of claim 9, wherein the IPTG is added at a concentration from 0.1 mM to 1 mM.

11. The method of claim 10, wherein IPTG is added only when the cell culture reaches an OD600 value of 30 or higher and induced for 10 hrs or longer.

12. The method of claim 8, wherein the first feed medium is added at a flow rate from 100 to 400 ml/hr, with stirring at a speed from 300 to 800 rpm.

13. The method of claim 12, wherein the feeding rate and the stirring speed are increased stepwise.

14. The method of claim 8, wherein the second feed medium is added at a flow rate from 50 to 300 ml/hr, with stirring at a speed from 300 to 800 rpm.

15. The method of claim 14, wherein the feeding rate is constant added and the stirring speed is decreased stepwise.

16. The method of claim 8, wherein the batch medium comprises:
(a) an organic carbon source selected from glucose, glycerol, fructose, lactose, sucrose, arabinose; galactose, and mannose;
(b) an organic nitrogen source selected from yeast extract and phytone peptone, which is animal source free and present as a component of the media or added to the media during fermentation; and
(c) an inorganic salt.

17. The method of claim 8, wherein the first feed medium comprising glucose at a concentration from 150 to 500 g/L and yeast extract at a concentration from 100 to 500 g/L.

18. The method of claim 8, wherein the second medium comprising glucose at a concentration from 50 to 400 g/L and yeast extract at a concentration from 50 to 400 g/L.

19. The method of claim 8, wherein the cell density has a final OD600 value of 60 or higher.

20. The method of claim 8, wherein the temperature during induction phase is shifted down from 37 degree Celsius to a temperature between 32-16 degree Celsius.

21. A method for purifying CRM197 recombinant protein from fermentation using the E. coli cell, comprising three steps of chromatography purification performed sequentially.

22. The method according to claim 21, wherein the first step of chromatograph comprises capture purification, which is performed by anionic exchange chromatography.

23. The method of claim 22, wherein the anion exchange chromatograph uses a resin selected diethylaminopropyl (ANX), diethyl-aminoethyl (DEAE), Quaternary aminoethyl (QAE), Quaternary ammonium (Q), positive charge function groups immobilized to a base matrix, or a mixed mode resin that comprises an N-benzyl-methyl ethanolamine functional group.

24. The method of claim 18, wherein the second step of chromatograph is performed by hydrophobic interaction chromatography (HIC).

25. The method of claim 24, where the HIC uses a resin comprising phenyl, butyl or octyl functional groups, wherein the aromatic or aliphatic ligands were immobilized to the base matrix.

26. The method of claim 21, the third step of chromatograph is performed by affinity chromatography.

27. The method of claim 5, comprising different functional affinity groups connected to the base matrix, wherein the functional affinity groups wherein said specified repeating dimer of hexuronic acid and D-glucosamine, or other ligands wherein Cibacron Blue 3G, Lysine, metal ion (Zn²⁺, Cu²⁺, Ni²⁺, Co₂₊ ions), lectins, calcium phosphate repeating stretch can be chosen.


SEQUENCE LISTING (3 sequences)

SEQ ID NO: 1 - DNA, 1605 bp, artificial
  synthetic
ggagccgatgacgtggttgacagctctaagtcgttcgttatggaaaacttcagttcctatcacggcaccaagccgggctacgtcgatagcattcagaaaggtatccaaaaaccgaagtctggcacgcagggtaactacgatgacgattggaaggaattttacagcaccgacaacaaatatgatgcggccggttacagtgttgacaacgaaaatccgctgtccggcaaggccggcggtgtcgtgaaagtgacgtatccgggcctgaccaaggttctggccctgaaagtcgataatgcagaaaccattaaaaaggaactgggtctgagcctgacggaaccgctgatggaacaagtgggcaccgaagaatttatcaaacgcttcggcgatggtgccagccgtgttgtcctgtctctgccgttcgcagaaggttcttcgagcgtcgaatatattaacaattgggaacaagcgaaggccctgagcgtggaactggaaatcaactttgaaacccgcggcaaacgtggtcaggatgcgatgtatgaatacatggcacaagcctgcgcgggtaatcgcgtgcgtcgctctgttggctctagtctgtcgtgtatcaacctggactgggatgttattcgtgataaaaccaagacgaaaattgaaagtctgaaggaacatggcccgatcaagaacaaaatgtctgaatcgccgaataaaacggtgtccgaagaaaaggcgaaacagtatctggaagaattccaccaaaccgcactggaacatccggaactgtcagaactgaaaaccgtcacgggtaccaacccggtgtttgccggcgcaaattacgcagcgtgggcagtcaacgtggcgcaagtgattgactcggaaacggccgataatctggaaaaaaccaccgccgcactgagcattctgccgggtatcggttctgtgatgggtattgccgatggcgcagttcatcacaacaccgaagaaattgtcgcccagagtatcgcactgtcctctctgatggttgcgcaagcgattccgctggtcggtgaactggtggatatcggctttgcagcgtataatttcgtggaatccattatcaacctgtttcaggtggttcacaacagttataatcgcccggcctactccccgggtcacaaaacccaaccgttcctgcatgacggctacgcggtgtcttggaatacggttgaagattcgattatccgtaccggctttcagggtgaatctggccacgacattaaaatcacggccgaaaacaccccgctgccgattgccggtgttctgctgccgaccatcccgggcaagctggatgtgaataagagtaaaacccatatctccgttaacggtcgcaaaattcgtatgcgctgccgtgcgatcgacggcgatgtgacgttctgtcgtccgaaaagcccggtttatgtcggcaacggtgttcatgcgaatctgcacgtcgcgtttcatcgttcgagctctgaaaaaattcatagcaacgaaatcagttccgattctattggtgtgctgggctaccagaaaaccgttgaccataccaaggtgaactctaaactgtctctgttctttgaaattaaatcc

SEQ ID NO: 2 - DNA, 1671 bp, artificial
  synthetic
atgaaatacctgctgccgaccgctgctgctggtctgctgctcctcgctgcccagccggcgatggccggagccgatgacgtggttgacagctctaagtcgttcgttatggaaaacttcagttcctatcacggcaccaagccgggctacgtcgatagcattcagaaaggtatccaaaaaccgaagtctggcacgcagggtaactacgatgacgattggaaggaattttacagcaccgacaacaaatatgatgcggccggttacagtgttgacaacgaaaatccgctgtccggcaaggccggcggtgtcgtgaaagtgacgtatccgggcctgaccaaggttctggccctgaaagtcgataatgcagaaaccattaaaaaggaactgggtctgagcctgacggaaccgctgatggaacaagtgggcaccgaagaatttatcaaacgcttcggcgatggtgccagccgtgttgtcctgtctctgccgttcgcagaaggttcttcgagcgtcgaatatattaacaattgggaacaagcgaaggccctgagcgtggaactggaaatcaactttgaaacccgcggcaaacgtggtcaggatgcgatgtatgaatacatggcacaagcctgcgcgggtaatcgcgtgcgtcgctctgttggctctagtctgtcgtgtatcaacctggactgggatgttattcgtgataaaaccaagacgaaaattgaaagtctgaaggaacatggcccgatcaagaacaaaatgtctgaatcgccgaataaaacggtgtccgaagaaaaggcgaaacagtatctggaagaattccaccaaaccgcactggaacatccggaactgtcagaactgaaaaccgtcacgggtaccaacccggtgtttgccggcgcaaattacgcagcgtgggcagtcaacgtggcgcaagtgattgactcggaaacggccgataatctggaaaaaaccaccgccgcactgagcattctgccgggtatcggttctgtgatgggtattgccgatggcgcagttcatcacaacaccgaagaaattgtcgcccagagtatcgcactgtcctctctgatggttgcgcaagcgattccgctggtcggtgaactggtggatatcggctttgcagcgtataatttcgtggaatccattatcaacctgtttcaggtggttcacaacagttataatcgcccggcctactccccgggtcacaaaacccaaccgttcctgcatgacggctacgcggtgtcttggaatacggttgaagattcgattatccgtaccggctttcagggtgaatctggccacgacattaaaatcacggccgaaaacaccccgctgccgattgccggtgttctgctgccgaccatcccgggcaagctggatgtgaataagagtaaaacccatatctccgttaacggtcgcaaaattcgtatgcgctgccgtgcgatcgacggcgatgtgacgttctgtcgtccgaaaagcccggtttatgtcggcaacggtgttcatgcgaatctgcacgtcgcgtttcatcgttcgagctctgaaaaaattcatagcaacgaaatcagttccgattctattggtgtgctgggctaccagaaaaccgttgaccataccaaggtgaactctaaactgtctctgttctttgaaattaaatcc

SEQ ID NO: 3 - protein, 535 aa, Corynebacterium diphtheriae
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS